(19) United States
(12) Patent Application Publication
(10) Pub. No.: US 2005/0101655 A1
(43) Pub. Date: May 12, 2005

(54) Migraine treatments including isovaleramide compounds and serotonin agonists

(76) Inventor: Linda D. Artman, Salt Lake City, UT (US)

Correspondence Address: TRASK BRITT, P.O. BOX 2550, SALT LAKE CITY, UT, 84110, US

(21) Appl. No.: 10/987,760

(22) Filed: Nov. 12, 2004

Related U.S. Application Data
(60) Provisional application No. 60/519,243, filed on Nov. 12, 2003.

(51) Int. Cl. A61K 31/405; A61K 31/16

(52) U.S. Cl. 514/419; 514/626

(57) ABSTRACT

A method of treating a migraine headache comprising administering at least one serotonin agonist and isovaleramide, α-methyl isovaleramide, or mixtures thereof to a patient suffering from a migraine. The at least one serotonin agonist is selected from the group consisting of sumatriptan, eleptriptan, naratriptan, rizatriptan, zolmitriptan, almotriptan, frovatriptan, ergotamine, an ergotamine derivative, and mixtures thereof. A migraine treatment is also provided.

### CROSS-REFERENCE TO RELATED APPLICATION

This application claims the benefit of U.S. Provisional Patent Application Ser. No. 60/519,243, filed Nov. 12, 2003, for MIGRAINE TREATMENTS INCLUDING ISOVALERAMIDE COMPOUNDS AND SEROTONIN AGONISTS, the disclosure of which is incorporated by reference in its entirety.

### FIELD OF THE INVENTION

The present invention relates to treatments of migraine headaches. More specifically, the present invention relates to migraine treatments that include at least one isovaleramide compound and at least one serotonin agonist.

### BACKGROUND OF THE INVENTION

Migraine headaches or migraines are estimated to affect approximately 28 million Americans and result in the loss of 64 million workdays annually. The symptoms of a migraine include a severe, pulsating headache that lasts from between 4 and 72 hours. In addition to the pain caused by the headache, other symptoms associated with the migraine include disturbances in vision, disturbances in mental and motor functions, fatigue, vomiting, nausea, cutaneous allodynia, cutaneous sensitivity, cutaneous hyperalgesia, osmophobia, photophobia, and/or phonophobia. These symptoms are referred to herein as the “associated migraine symptoms.” The term “migraine symptoms” is used herein to refer to both the headache and the associated migraine symptoms. Cutaneous allodynia refers to pain that is elicited in a migraine sufferer by a stimulus that would typically not elicit pain in a non-migraine individual. Normal nonnoxious stimuli, such as wearing clothing, earrings, contact lenses or glasses, shaving, brushing hair, or contacting warm or cool air, are described by migraine sufferers as extremely painful during the migraine episode.

According to the US Headache Consortium, successful treatment of acute migraines includes the following series of broad strategies and outcomes: treat attacks rapidly and consistently without recurrence; restore the patient's ability to function; minimize the use of backup and rescue medications; optimize self-care and reduce subsequent use of resources; be cost-effective for overall management; and have minimal or no adverse effects. Matchar et al., U.S. Headache Consortium, Evidence-based guidelines for migraine headache in the primary care setting: pharmacological management of acute attacks, American Academy of Neurology (April 2000).

The migraine is typically triggered by various factors, such as hormonal changes, stress, foods, lack of sleep, excessive sleep, or visual, auditory, olfactory or somatosensory stimulation. The exact mechanism of migraine initiation and progression is not known. However, the migraine typically progresses through four phases: a prodrome phase, in which the migraine sufferer experiences lethargy, euphoria, food cravings, depression, or irritability; an aura phase in which the migraine sufferer has visual changes, numbness, or dizziness; a headache phase lasting from 4-72 hours; and a postdrome phase in which the headache is reduced but many of the other migraine symptoms remain.

While many hypotheses describing initiation and maintenance of the migraine have been proposed, one current hypothesis is that an initial stimulus or episodic phenomenon activates sensory nerves that innervate intracranial blood vessels, extracranial blood vessels, and the meninges. Bendtsen, Current Opinions in Investigational Drugs 3(3):449-453 (2002). The initial stimulus causes vasodilatation of the intracranial blood vessels, extracranial blood vessels, and the meninges, which projects pain to portions of the brain. Id. The vasodilatation is believed to sensitize pain pathways, which are referred to as “peripheral sensitization” and “central sensitization.” Id. During peripheral sensitization, first order neurons in the CNS, such as trigeminal sensory neurons, are sensitized. Peripheral sensitization is believed to be involved in the initiation of the migraine. Id.

Further sensitization of second and third order neurons, such as of medullary dorsal horn neurons, is believed to cause central sensitization, which maintains and exacerbates the migraine. Id. Initiation and maintenance of central sensitization are thought to be distinct phases of the migraine. Burstein, Pain 89:107-110 (2001). The initiation of central sensitization is caused by neuronal input from the periphery while central sensitization is maintained with no further neuronal input from the periphery. Id. Symptoms of central sensitization include cutaneous allodynia or increased cutaneous sensitivity and hyperalgesia, which have recently been recognized as important phenomena in migraines. These symptoms associated with central sensitization typically occur at a late phase in the migraine progression compared to other symptoms. The current migraine therapies, such as the triptans, do not relieve the symptoms of central sensitization and, therefore, migraine sufferers experience inadequate pain relief during the course of their migraine. Rather, the current migraine therapies typically provide relief for symptoms that occur at an early phase of the migraine.

Current migraine treatments are categorized as prophylactic treatments or as abortive treatments. Each category of treatments is administered to the migraine sufferer based on the frequency and severity of the headache and its associated symptoms. Prophylactic treatments reduce the frequency of migraines and include non-steroidal anti-inflammatory agents, andrenergic beta-blockers, calcium channel blockers, tricyclic antidepressants, selective serotonin reuptake inhibitors, or anticonvulsants. For occasional migraines, abortive treatments are used. The abortive treatments eliminate or reduce the severity of the headache and any associated symptoms after the migraine has begun. Abortive treatments include serotonin receptor agonists, such as triptan compounds or ergotamine-based compounds, or compounds that provide analgesic effects. However, sumatriptan and other triptan compounds are only reported to relieve the migraine symptoms in approximately 40% of patients and do not relieve symptoms that typically occur at late stages of the migraine, such as during central sensitization.

Central sensitization has been documented as a cause of somatosensory hypersensitivity observed in patients after surgical trauma. Woolf, Nature, 306:686-688 (1983). Central sensitization begins with the induction of activity in peripheral C fibers, which can be activated by mechanical, thermal, or chemical stimuli. Dahl et al., Br. J. Anaesth., 69:117-121 (1992); Tasker et al., J. Neurosurg., 77:373-378 (1992). There is scientific evidence that central sensitization is also produced in the trigeminal system and that it has a particular role in the pathogenesis of migraine. Burstein and colleagues used quantitative sensory testing to study changes in mechanical and thermal thresholds of periorbital and forearm skin areas to determine sensitivity during migraine attacks. Burstein et al., Basic science of the headaches: central sensitization and headache. In: The Headache, 2^{nd} Ed., Philadelphia, Pa., Lippincott Williams & Wilkins (2000). Seventy-five percent of the subjects studied exhibited cutaneous allodynia during their migraine. These investigators concluded that the existence of cutaneous allodynia suggested that the pathophysiology of migraine involves not only peripheral neurons, but also central brainstem and thalamic neurons. Because central sensitization is poorly treated by traditional migraine therapies (i.e., triptans), it is likely that drugs that reduce central sensitization will show a reduction in cutaneous allodynia, which develops in the majority of migraine headaches. Cutaneous allodynia is considered to be a marker of the development of central sensitization during a migraine, which correlates with patient observations that triptan migraine therapy is more effective if taken early in the migraine cascade before central sensitization, i.e., cutaneous allodynia, develops. None of the currently available therapies effectively treat the migraine once cutaneous allodynia is present. The presence or absence of cutaneous allodynia may become a marker by which patients will decide if conventional migraine therapies effectively treat the migraine.

Many anticonvulsant compounds, e.g., topiramate, valproic acid, gabapentin, levetiracetam, and carbamazepine, used in the treatment of a wide range of seizure disorders are also effective prophylactic migraine treatments. While the mechanism by which these anticonvulsant compounds alleviate the migraine is not known, it is believed that the anticonvulsant compounds stabilize episodic phenomena from various biochemical or physical origins and generally result in a decrease in central nervous system (“CNS”) excitability. Many of these anticonvulsant compounds, such as valproic acid, do not have a rapid oral bioavailability and, therefore, are not effective abortive treatments when administered orally. However, valproic acid is effective as an abortive treatment when administered intravenously, although intravenous treatments are less desirable than oral treatments.

The effectiveness of many of the migraine treatments depends on the phase in which the treatment is administered. The prophylactic treatments are more effective when administered chronically and before the migraine occurs, while the abortive treatments are administered at the time the migraine occurs, such as during the headache phase. Once the migraine has actually occurred, if the headache and its associated symptoms are not treated early in the progression of the migraine, then patients report that the symptoms progress and worsen compared to a migraine that is treated earlier or the patients typically do not experience complete pain relief.

Isovaleramide has analgesic effects and has been disclosed as a treatment for affective mood disorders, acute muscular aches, muscle strains, muscle sprains, chronic headaches, cluster headaches, migraine headaches, restlessness syndromes, neuropathic pain, movement disorders, spasticity, convulsions, cerebral insult, neurodegeneration, and substance abuse. Isovaleramide has anticonvulsant activity that is similar in breadth to that of valproic acid and reduces the symptoms associated with peripheral sensitization (e.g., cutaneous stimulation-induced hyperreflexia) without exhibiting the serious adverse side effects that are observed with valproic acid, such as hepatotoxicity, teratogenicity, or pancreatitis. In addition, isovaleramide is rapidly absorbed, reaching near peak blood levels within 15 minutes post dosing orally.

Therefore, it would be desirable to provide a migraine therapy to reduce cutaneous allodynia and that is effective in aborting the entire spectrum of migraine phenomena throughout the time period of a migraine occurrence.

### BRIEF SUMMARY OF THE INVENTION

The present invention relates to methods of treating a migraine. The method comprises administering isovaleramide, α-methyl isovaleramide, or mixtures thereof to a patient suffering from a migraine and administering at least one serotonin agonist, such as a triptan compound or an ergotamine compound, to the patient. The at least one serotonin agonist may be selected from the group consisting of sumatriptan, eleptriptan, naratriptan, rizatriptan, zolmitriptan, almotriptan, frovatriptan, ergotamine, an ergotamine derivative, and mixtures thereof. The at least one serotonin agonist and the isovaleramide, α-methyl isovaleramide, or mixtures thereof may be administered to the patient in a single pharmaceutical composition. Alternatively, the at least one serotonin agonist and the isovaleramide, α-methyl isovaleramide, or mixtures thereof may be coadministered to the patient. The at least one serotonin agonist and the isovaleramide, α-methyl isovaleramide, or mixtures thereof may reduce or alleviate the symptoms associated with the migraine, such as the symptoms associated with central sensitization.

The present invention also relates to a migraine treatment comprising at least one serotonin agonist and isovaleramide, α-methyl isovaleramide, or mixtures thereof. The at least one serotonin agonist may be selected from the group consisting of sumatriptan, eleptriptan, naratriptan, rizatriptan, zolmitriptan, almotriptan, frovatriptan, ergotamine, an ergotamine derivative, and mixtures thereof.

### BRIEF DESCRIPTION OF THE SEVERAL VIEWS OF THE DRAWINGS

While the specification concludes with claims particularly pointing out and distinctly claiming that which is regarded as the present invention, the advantages of this invention may be more readily ascertained from the following description of the invention when read in conjunction with the accompanying drawings in which:

FIG. 1 illustrates the analgesic effect of isovaleramide in a hot plate test in mice compared to the effect of a vehicle or morphine;

FIG. 2 illustrates the effect of 300 mg/kg isovaleramide on observational scores of hyperreflexia elicited by nonnoxious stimuli in chronic spinalized rats;

FIG. 3 illustrates a time-dependent reduction of the flexor reflex, an electrophysiological assessment of pain and muscle tone, in the chronic spinalized rats following treatment with isovaleramide (NPS 1776); and

FIG. 4 shows a dose-response relationship on the flexor reflex for isovaleramide (NPS 1776) and baclofen, a known muscle relaxant with analgesic properties.

### DETAILED DESCRIPTION OF THE INVENTION

A treatment for a migraine is provided. The migraine treatment is a pharmaceutical composition that includes at least one serotonin agonist and isovaleramide, α-methyl isovaleramide, or mixtures thereof. The pharmaceutical composition reduces or eliminates the symptoms of central sensitization and other migraine symptoms that may occur. Since the pharmaceutical composition provides relief from symptoms that occur at early or late phases of the migraine, effective relief may be provided to a migraine sufferer throughout the duration of the migraine.

Isovaleramide has the following structure: 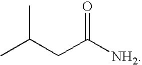
Isovaleramide has a bioavailability of approximately 100% and a quick onset of action. For instance, near peak blood levels of isovaleramide are observed approximately 15 minutes after the isovaleramide is administered orally. Compounds that are structurally related to isovaleramide, such as α-methyl isovaleramide, may also be used in the pharmaceutical composition. α-methyl isovaleramide has the following structure and has been shown to have anticonvulsant activity: 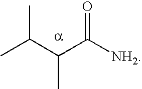
In addition, mixtures of isovaleramide and α-methyl isovaleramide may be used in the pharmaceutical composition. Isovaleramide, α-methyl isovaleramide, or mixtures thereof are referred to herein as an “isovaleramide compound.”

The isovaleramide compound may be isolated from a valerian extract, prepared by a synthetic route, or a combination thereof, as described in U.S. Pat. No. 6,589,994, which is incorporated by reference herein. Preferably, the isovaleramide compound is prepared by conventional synthetic techniques. For example, carboxylic acid precursors of isovaleramide or α-methyl isovaleramide, which are available from Sigma-Aldrich Chemical Co. (St. Louis, Mo.), may be used. The carboxylic acid precursor may be converted into the corresponding amide by preparation of the acid chloride with thionyl chloride or oxalyl chloride, followed by reaction with ammonia or an amine. If a carboxylic acid precursor is not commercially available, the carboxylic acid precursor may be prepared by conventional organic synthetic techniques. For example, carboxylic acid esters may be deprotonated with a strong non-nucleophilic base, such as lithium diisopropylamide, followed by alkylation with methyl iodide or methyl trifluoromethanesulfonate. The alkylated ester may then be hydrolyzed and converted to the amide by the methods described above.

Enantiomers of the α-methyl isovaleramide may be prepared from optically active starting materials. Alternatively, the individual enantiomers may be separated by conventional methods of resolution, such as fractional crystallization of salts with chiral amines, or by preparation of amides with chiral amides, chromatographic separation, and hydrolysis of the amides. α-methyl isovaleramide may also be prepared by conventional techniques of asymmetric synthesis, such as by alkylation of an ester or amide of the acid prepared using a chiral auxiliary.

The pharmaceutical composition may also include a serotonin agonist, such as a 5-HT_{1B/1D} agonist, a 5-HT_{1B/1F} agonist, a 5HT_{1D/1F} agonist, a 5HT_{1D} agonist, a 5HT_{1F} agonist, or an ergotamine compound. The serotonin agonist may be a triptan compound, including, but not limited to, sumatriptan, eleptriptan, naratriptan, rizatriptan, zolmitriptan, almotriptan, frovatriptan, and mixtures thereof. The triptan compound may be obtained through a commercial source, such as GlaxoSmithKline, Pfizer Corp., Merck & Co., Inc., AstraZeneca Pharmaceuticals LP, Ortho-McNeil Pharmaceuticals, or Elan Pharmaceuticals Inc., or it may be synthesized by conventional techniques. The ergotamine compound may be ergotamine or an ergotamine derivative. The ergotamine compound may be obtained through a commercial source or may be synthesized by conventional techniques.

It is also contemplated that the pharmaceutical composition may include a serotonin agonist in combination with an anticonvulsant compound having anticonvulsant activity, such as levetiracetam, pregabalin, or valproic acid.

The pharmaceutical composition including the isovaleramide compound and at least one serotonin agonist may be prepared by conventional techniques. For instance, the isovaleramide compound and the serotonin agonist may be combined with a pharmaceutically acceptable carrier and formed into the pharmaceutical composition. The pharmaceutically acceptable carrier may be any suitable carrier whose administration is tolerated by the patient. Pharmaceutically acceptable carriers, such as solutions or excipients, are known in the art and include, but are not limited to, sterile phosphate-buffered saline, saline, or Ringer's solutions. However, any other pharmaceutically acceptable carriers may also be used.

The pharmaceutical composition may be administered to a patient in need of migraine treatment in a therapeutically effective amount. As used herein, the term “therapeutically effective amount” refers to a total amount of the isovaleramide compound and of the serotonin agonist that results in a detectable change in the severity of the patient's migraine symptoms. The therapeutically active amount may provide a concentration of the isovaleramide compound and of the serotonin agonist to the patient that is pharmacologically active and pharmaceutically effective. The patient suffering from the migraine may exhibit at least one of the migraine symptoms previously mentioned, such as the headache, disturbances in vision, disturbances in mental and motor functions, fatigue, vomiting, nausea, cutaneous allodynia, cutaneous sensitivity, cutaneous hyperalgesia, osmophobia, photophobia, or phonophobia. The amount or dose of the isovaleramide compound and the serotonin agonist in the pharmaceutical composition may be based on the patient's age, weight, height, sex, general medical condition, and previous medical history, as known in the art.

The pharmaceutical composition may be administered orally using a solid oral dosage form, such as an uncoated tablet, an enteric-coated tablet, a film-coated tablet, a caplet, a gelcap, a capsule, or a powder. The pharmaceutical composition may also be administered as a liquid oral dosage form, such as a syrup, tincture, concentrate, or elixir. The dosage of the isovaleramide compound used to reduce the migraine symptoms may range from approximately 50 mg per dose to approximately 2400 mg per dose. Unit solid oral dosage forms may, for example, include about approximately 200 mg to approximately 600 mg of the isovaleramide compound per tablet or capsule, at a dosage ranging from approximately 1 mg/kg body weight to approximately 20 mg/kg body weight. Liquid formulations may also be employed, preferably admixed to provide a suitable dose in 1 or 2 teaspoonfuls for convenient administration. Reduced dosage pediatric chewable and liquid oral dosage forms may also be prepared and administered. The isovaleramide compound and the serotonin agonist may also be added to foods and beverages in the form of drops (with a dropper from a concentrated preparation of the pharmaceutical composition) for oral administration. In addition, the isovaleramide compound and the serotonin agonist may be formulated into chewing gum to facilitate oral delivery and absorption. A dosage of the serotonin agonist that is effective to reduce the migraine symptoms is known in the art. For instance, unit solid oral dosage forms may include from approximately 1 mg to approximately 100 mg of the serotonin agonist.

Depending on the oral bioavailability of the isovaleramide compound and the serotonin agonist in the pharmaceutical composition, each of these compounds may be delivered to the patient essentially simultaneously. However, the isovaleramide compound and the serotonin agonist may be delivered to the patient at substantially different times as long as the therapeutically effective amount of the isovaleramide compound and the serotonin agonist is achieved in the patient.

In addition to oral administration, the pharmaceutical composition may be administered by injection or another systemic route, such as by intravenous, transdermal, or transmucosal administration. For instance, the pharmaceutical composition may be administered nasally, buccally, or rectally.

The pharmaceutical composition may be formulated into a single pharmaceutical composition so that the isovaleramide compound and the serotonin agonist are essentially simultaneously released from the pharmaceutical composition and are delivered to the patient essentially simultaneously. The pharmaceutical composition may also be formulated into a single pharmaceutical composition so that the isovaleramide compound and the serotonin agonist are released from the pharmaceutical composition at substantially different times but still deliver the therapeutically effective amount of the isovaleramide compound and the serotonin agonist.

The isovaleramide compound and the serotonin agonist may also be coadministered to the patient. As used herein the term “coadministered” refers to separate administration of the isovaleramide compound and the serotonin agonist to the patient so that the therapeutically effective amount of the isovaleramide compound and the serotonin agonist is available to or present in the patient. For instance, the isovaleramide compound and the serotonin agonist may be separately administered at times that are sufficiently close together to provide the therapeutically effective amount of the isovaleramide compound and the serotonin agonist. For sake of example only, the serotonin agonist may be administered to the patient first, followed by administration of the isovaleramide compound. The isovaleramide compound may be administered to the patient within a sufficient amount of time after the administration of the serotonin agonist to provide the therapeutically effective amount of the isovaleramide compound and the serotonin agonist.

The pharmaceutical composition may be administered to the patient after initiation of the migraine. For instance, the patient may be in the headache phase of the migraine or the postdrome phase before the pharmaceutical composition is administered. Alternatively, the pharmaceutical composition may be administered to the patient before the migraine starts, such as once the patient senses that a migraine is approaching or when the early symptoms of the migraine have begun. Without being tied to a particular theory, it is believed that the pharmaceutical composition reduces or eliminates the migraine symptoms because the serotonin agonist elicits vasoconstriction of the dilated cranial arteries and the isovaleramide compound alters the phenomenon of central sensitization. The serotonin agonist and the isovaleramide compound also provide analgesic benefits to the patient. Therefore, the pharmaceutical composition may provide effective relief throughout the migraine episode, including relieving those symptoms that occur during central sensitization. For instance, the pharmaceutical composition may reduce or eliminate the pain associated with cutaneous allodynia.

The pharmaceutical composition including the combination of the isovaleramide compound and the serotonin agonist may more effectively reduce or eliminate the migraine symptoms compared to a treatment that includes either the isovaleramide compound or the serotonin agonist alone. The pharmaceutical composition may provide more complete relief from the migraine symptoms because it affects more of the migraine symptoms than a treatment that includes either the isovaleramide compound or the serotonin agonist alone. The pharmaceutical composition may be particularly effective to reduce or eliminate the migraine symptoms that occur during central sensitization.

Therapeutic activity of the pharmaceutical composition may be determined in various animal models of neuropathic pain or in clinically relevant studies of different types of neuropathic pain. The therapeutic activity may be determined without determining a specific mechanism of action. Animal models for neuropathic pain are known in the art and include, but are not limited to, animal models that determine analgesic activity or compounds that act on the CNS to reduce the phenomenon of central sensitization that results in pain from nonpainful or nonnoxious stimuli. Other animal models that are known in the art, such as hot plate tests, model acute pain and are useful for determining analgesic properties of compounds that are effective when painful or noxious stimuli are present. The progression of migraines is believed to be similar to the progression of epilepsy (because an episodic phenomenon underlies the initiation of the epileptic episode) and, as such, it is believed that epilepsy animal models may be useful in determining a component of the therapeutic activity of the pharmaceutical composition. The therapeutic activity of the pharmaceutical composition may also be determined in animal models of migraine.

### EXAMPLES

### Example 1

### Analgesic Activity of Isovaleramide

Isovaleramide was administered orally at 600 mmol/kg to ten mice. Morphine was administered as a reference substance at 64 mg/kg to ten mice under the same experimental conditions. A vehicle was administered to ten mice as a control substance under the same experimental conditions. The isovaleramide, morphine, or vehicle was administered to the mice in a blind study. Sixty minutes after the isovaleramide, morphine, or vehicle were administered, the mice were placed onto a hot metal plate maintained at 54° C. and surrounded by a Plexiglass cylinder, according to the method of Eddy and Leimbach. See Eddy et al., J. Pharmacol. Exp. Ther. 107: 385-393 (1953). The time taken for the mice to lick their feet is an index of analgesic activity. Effective analgesics increase the latency or amount of time to licking. Latency to the first foot lick was measured, up to a maximum time of 30 seconds to prevent tissue damage to the mice.

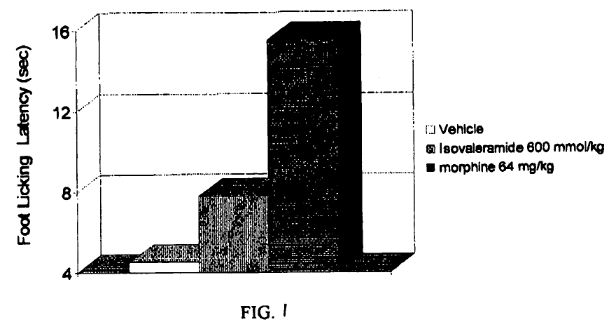

As shown in FIG. 1, mice that received the isovaleramide had an increased foot licking latency compared to the mice that received the vehicle. This animal model may also be used to demonstrate the analgesic effects of isovaleramide alone or in combination with the serotonin agonist, whether the effect is additive or synergistic.

### Example 2

### Effects of Isovaleramide in Hyperreflexia and Flexor Reflex Tests

Assessment of hyperreflexia, pain, and muscle tone in chronic spinally transected rats was conducted using male albino Holtzman-derived rats (available from Harlan Sprague-Dawley Laboratories) weighing 270-530 grams as subjects. The rats were housed independently and had continuous access to food and water throughout the experiments. All procedures were reviewed and approved by the Institutional Animal Care and Use Committee. Animals were anesthetized using a mixture of isoflurane and oxygen at a flow rate of 4 liters/minute.

The rats were placed in a stereotaxic frame and anesthesia was maintained. An incision was made so that the paraspinal muscles could be retracted and a laminectomy performed between T6-T9. A one- to two-millimeter portion of the spinal cord was removed by evacuation and replaced with gel foam to reduce bleeding, after which the incision was closed in layers.

Following the transection, rats were placed in a room in which the ambient temperature was raised to about 80° F. with a space heater to maintain body temperature. On the following morning post-surgery, the hindquarters of the spinalized rats were bathed and their urine expressed manually by applying pressure to their bladders. Experiments were conducted between 21 and 28 days after surgery. For the first two weeks post-surgery, 0.25 ml of the antibiotic Sulfatrim Pediatric Suspension was orally administered to the rats to prevent bladder infection. A commercial antibiotic cream was applied to any part of the skin that showed signs of decubitus lesions. Within approximately two weeks, all animals regained bladder control and were no longer given antibiotic treatment. Assessment of hyperreflexia and flexor reflex was performed before and after drug treatment so that each animal served as its own control.

Initial assessment of hyperreflexia was performed by the subjective scoring method of rating the resulting hyperreflexia response elicited with an innocuous stimulus, such as a metal probe. The metal probe was pressed against the lower abdomen at four specific sites. The response was evaluated for each of four trials using a scale ranging from zero (no response in all four trials) to four (a maximum, tonic-clonic reaction elicited in all four trials). All scores, pre- and post-treatment, were transformed to indicate the percent of hyperreflexia, pain, or muscle tone so that a score of 0/4=0%, 1/4=25%, etc. The raw or normalized scores were analyzed with a one-way repeated measures ANOVA. Three rats were tested per group.

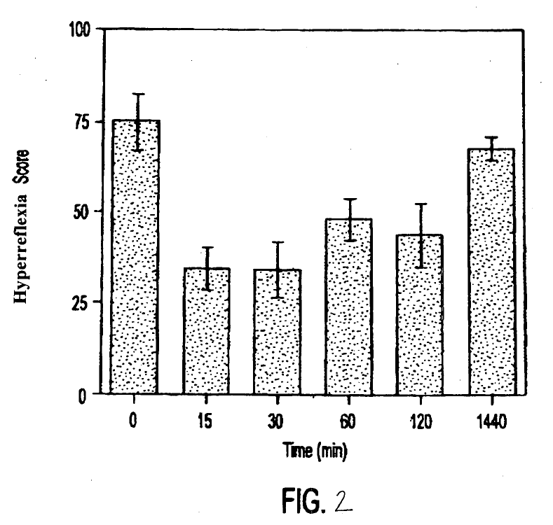

After determining the assessment of hyperreflexia before drug treatment, 300 mg/kg of isovaleramide was administered intraperitoneally (“i.p.”) to the rats. As shown in FIG. 2, isovaleramide at a dose of 300 mg/kg, i.p., was efficacious at 15, 30, 60, and 120 minutes post-administration in reducing the scores (45-65%). The bar at time zero represents pre-treatment control values. By the next day, i.e., by 1440 minutes (24 hours), the scores had essentially returned to baseline values. No overt behavioral toxicity or motor impairment was observed at this dose. The rats were alert and able to grasp with their non-paralyzed front paws as were the untreated control rats.

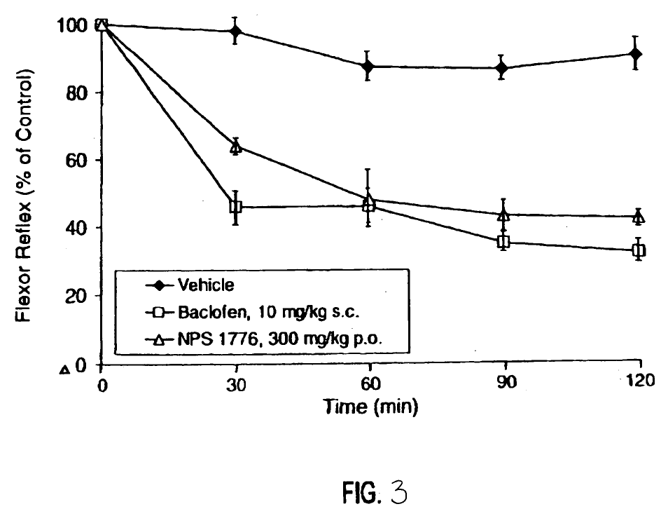

With reference to FIG. 3, polysynaptic flexor-reflex responses, elicited by stimuli that activate high-threshold afferents, were recorded as EMG activity from the ipsilateral hamstring muscle. Supramaximal electric shocks were applied to the hindpaw and recording electrodes were placed in the biceps femoris semitendinosus muscle. Five sets of stimuli were made at each time point. The flexor reflex was recorded, in both the pre-drug and the post-drug periods, every 30 minutes once a stable baseline response was achieved. The data at time zero represent pre-treatment control values. The responses were determined in spinalized rats by observing the flexor-reflex response before treatment and at each of 30, 60, 90, and 120 minutes following administration of isovaleramide (300 mg/kg p.o.), baclofen (10 mg/kg s.c.) and vehicle (water, 12 ml/kg p.o.), respectively. Isovaleramide was shown to reduce the magnitude of the flexor-reflex responses in a chronic spinalized rat at all time points with similar efficacy to baclofen, the positive control.

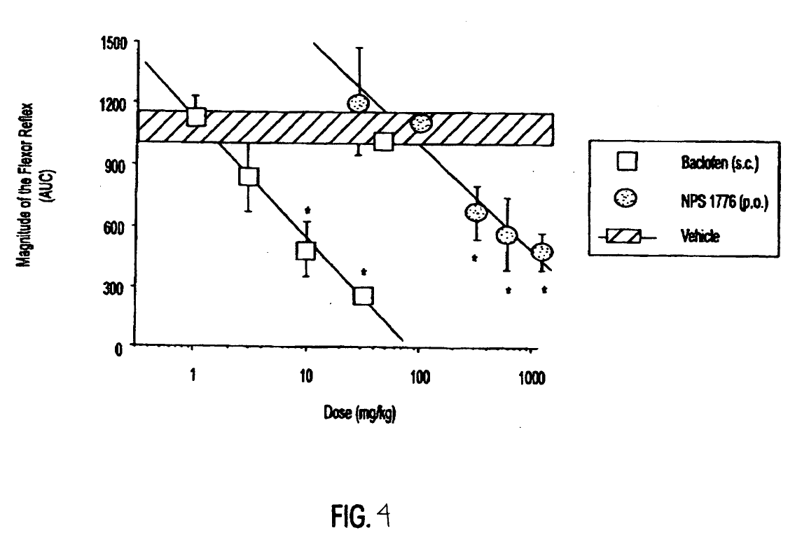

In FIG. 4, the responses from FIG. 3 and additional doses of isovaleramide and baclofen were converted to a total-area-under-the-curve format, covering the entire, two-hour measurement period. All drug-treated groups differed significantly from the vehicle (p<0.05), based on a one-way analysis of variance (ANOVA). Between the drug-treated groups, no differences were found in total reduction of the flexor reflex over the two-hour period (pairwise multiple comparison, Student-Newman-Keuls method). In other words, isovaleramide and baclofen produced a similar dose-dependent reduction of the flexor reflex in the chronic spinalized rat.

### Example 3

### Effects of Isovaleramide and Sumatripan in Cutaneous Hypersensitivity Tests

The effects of isovaleramide and sumatriptan on nociceptive activation of the trigeminovascular system are determined using the migraine model described in Goadsby et al., Adenosine A1 receptor agonists inhibit trigeminovascular nociceptive transmission, Brain, 125:1392-1401 (2002). A pharmaceutical composition including from 1 mg/kg to 1000 mg/kg of isovaleramide and from 3 μg/kg to 1000 μg/kg of sumatriptan is administered to cats. To serve as positive and negative controls, a vehicle control or individual compositions of isovaleramide or sumatriptan are administered to the cats.

The cats that receive the combination of the isovaleramide and sumatriptan have inhibited trigeminovascular activation compared to the trigeminovascular activation in the cats that receive the vehicle. The cats receiving the combination of the isovaleramide and sumatriptan also have inhibited trigeminovascular activation compared to the trigeminovascular activation of the cats that receive either the isovaleramide alone or the sumatriptan alone.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A method of treating a migraine, comprising:
administering isovaleramide, α-methyl isovaleramide, or mixtures thereof to a patient suffering from a migraine; and
administering at least one serotonin agonist to the patient.

2. The method of claim 1, wherein administering at least one serotonin agonist to the patient comprises administering at least one serotonin agonist selected from the group consisting of a 5-HT_{1B/1D} agonist, a 5-HT_{1B/1F} agonist, a 5HT_{1D/1F} agonist, a 5HT_{1D} agonist, a 5HT_{1F} agonist, and mixtures thereof to the patient.

3. The method of claim 1, wherein administering at least one serotonin agonist to the patient comprises administering at least one serotonin agonist selected from the group consisting of sumatriptan, eleptriptan, naratriptan, rizatriptan, zolmitriptan, almotriptan, frovatriptan, ergotamine, an ergotamine derivative, and mixtures thereof to the patient.

4. The method of claim 1, wherein administering isovaleramide, α-methyl isovaleramide, or mixtures thereof to a patient suffering from a migraine and administering at least one serotonin agonist to the patient comprises administering a pharmaceutical composition comprising isovaleramide, α-methyl isovaleramide, or mixtures thereof and the at least one serotonin agonist to the patient.

5. The method of claim 1, wherein administering isovaleramide, α-methyl isovaleramide, or mixtures thereof to a patient suffering from a migraine and administering at least one serotonin agonist to the patient comprises coadministering isovaleramide, α-methyl isovaleramide, or mixtures thereof and the at least one serotonin agonist to the patient.

6. The method of claim 1, further comprising reducing or alleviating symptoms associated with the migraine.

7. The method of claim 6, wherein reducing or alleviating symptoms associated with the migraine comprises reducing or alleviating the symptoms associated with central sensitization.

8. The method of claim 1, wherein administering isovaleramide, α-methyl isovaleramide, or mixtures thereof to a patient suffering from a migraine and administering at least one serotonin agonist to the patient comprises administering a migraine composition to a patient, the migraine composition comprising isovaleramide, α-methyl isovaleramide, or mixtures thereof and at least one serotonin agonist.

9. The method of claim 1, wherein the at least one serotonin agonist is administered prior to administering the isovaleramide, α-methyl isovaleramide, or mixtures thereof.

10. The method of claim 1, wherein administering isovaleramide, α-methyl isovaleramide, or mixtures thereof to a patient suffering from a migraine and administering at least one serotonin agonist to the patient comprises administering isovaleramide, α-methyl isovaleramide, or mixtures thereof and the at least one serotonin agonist as an oral dosage form selected from the group consisting of an enteric-coated tablet, an uncoated tablet, a film-coated tablet, a caplet, a gelcap, and a capsule.

11. The method of claim 1, wherein administering isovaleramide, α-methyl isovaleramide, or mixtures thereof to a patient suffering from a migraine and administering at least one serotonin agonist to the patient comprises administering isovaleramide, α-methyl isovaleramide, or mixtures thereof and the at least one serotonin agonist as a liquid dosage form selected from the group consisting of a syrup, a tincture, a concentrate, and an elixir.

12. The method of claim 1, wherein administering isovaleramide, α-methyl isovaleramide, or mixtures thereof to a patient suffering from a migraine and administering at least one serotonin agonist to the patient comprises administering isovaleramide, α-methyl isovaleramide, or mixtures thereof and the at least one serotonin agonist orally, intraperitoneally, intravenously, rectally, nasally, or buccally.

13. The method of claim 1, wherein the isovaleramide, α-methyl isovaleramide, or mixtures thereof is administered during an early phase of the migraine.

14. The method of claim 1, wherein the isovaleramide, α-methyl isovaleramide, or mixtures thereof is administered during a late phase of the migraine.

15. The method of claim 1, wherein the at least one serotonin agonist is administered during the early phase of the migraine.

16. The method of claim 1, wherein the at least one serotonin agonist is administered during the late phase of the migraine.

17. A migraine treatment comprising at least one serotonin agonist and isovaleramide, α-methyl isovaleramide, or mixtures thereof.

18. The migraine treatment of claim 17, wherein the at least one serotonin agonist is selected from the group consisting of a 5-HT_{1B/1D} agonist, a 5-HT_{1B/1F} agonist, a 5HT_{1D/1F} agonist, a 5HT_{1D} agonist, a 5HT_{1F} agonist, and mixtures thereof.

19. The migraine treatment of claim 17, wherein the at least one serotonin agonist is selected from the group consisting of sumatriptan, eleptriptan, naratriptan, rizatriptan, zolmitriptan, almotriptan, frovatriptan, ergotamine, an ergotamine derivative, and mixtures thereof.

20. The migraine treatment of claim 17, wherein the migraine treatment is formulated as an oral dosage form selected from the group consisting of an enteric-coated tablet, an uncoated tablet, a film-coated tablet, a caplet, a gelcap, and a capsule.

21. The migraine treatment of claim 17, wherein the migraine treatment is formulated as a liquid dosage form selected from the group consisting of a syrup, a tincture, a concentrate, and an elixir.

22. The migraine treatment of claim 17, wherein the migraine treatment is formulated to be administered orally, intraperitoneally, intravenously, rectally, nasally, or buccally.

23. The migraine treatment of claim 17, wherein the at least one serotonin agonist is present from approximately 1 mg to approximately 500 mg.

24. The migraine treatment of claim 17, wherein the isovaleramide, α-methyl isovaleramide, or mixtures thereof is present from approximately 50 mg to approximately 2400 mg.
